(19) United States
(12) Patent Application Publication
(10) Pub. No.: US 2022/0304605 A1
(43) Pub. Date: Sep. 29, 2022

(54) BIOMAGNETIC MEASUREMENT APPARATUS

(71) Applicants: Takanobu OSAKA, Hokkaido (JP); Hironobu MIFUNE, Miyagi (JP)

(72) Inventors: Takanobu OSAKA, Hokkaido (JP); Hironobu MIFUNE, Miyagi (JP)

(73) Assignee: Ricoh Company, Ltd., Tokyo (JP)

(21) Appl. No.: 17/654,868

(22) Filed: Mar. 15, 2022

(30) Foreign Application Priority Data
Mar. 23, 2021 (JP) 2021-048580

(51) Int. Cl. A61B 5/245; A61B 5/00

(52) U.S. Cl. CPC A61B 5/245; A61B 5/683; A61B 2562/0223; A61B 2562/046

(57) ABSTRACT

A biomagnetic measurement apparatus including: a magnetic sensor; a sensor holder having a cylindrical shape and configured to accommodate and hold the magnetic sensor; a sensor fixing part to which the sensor holder is fixed; and a base to which the sensor fixing part is mounted and configured to be mounted to an object of measurement. The sensor holder includes: a pressing part configured to press the magnetic sensor toward the object of measurement; and a plate including a protrusion configured to engage with the sensor fixing part.

### CROSS-REFERENCE TO RELATED APPLICATIONS

The present application is based on and claims priority to Japanese Patent Application No. 2021-048580 filed on Mar. 23, 2021, the contents of which are incorporated herein by reference in their entirety.

### BACKGROUND OF THE INVENTION

### Field of the Invention

The disclosures herein generally relate to a biomagnetic measurement apparatus.

### Description of the Related Art

As a biological signal measurement apparatus for measuring a biological signal such as a magnetic field emitted from a living body, a magnetoencephalography (MEG), a magnetocardiography (MCG), a magnetospinography (MSG), and the like are known.

In order to perform measurement using a biological signal measurement apparatus, it is necessary to attach a sensor used in the biological signal measurement apparatus to an object of measurement.

### SUMMARY OF THE INVENTION

According to an embodiment, a biomagnetic measurement apparatus includes: a magnetic sensor; a sensor holder having a cylindrical shape and configured to accommodate and hold the magnetic sensor; a sensor fixing part to which the sensor holder is fixed; and a base to which the sensor fixing part is mounted and configured to be mounted to an object of measurement, wherein the sensor holder includes: a pressing part configured to press the magnetic sensor toward the object of measurement; and a plate including a protrusion configured to engage with the sensor fixing part.

### BRIEF DESCRIPTION OF THE DRAWINGS

FIG. 1 is a diagram illustrating a state of use of a biomagnetic measurement apparatus according to the present embodiment;

FIG. 2 is a diagram illustrating a mounting part of the biomagnetic measurement apparatus according to the present embodiment;

FIG. 3 is a diagram illustrating a sensor fixing part of the biomagnetic measurement apparatus according to the present embodiment;

FIG. 4 is a diagram illustrating a sensor assembly of the biomagnetic measurement apparatus according to the present embodiment;

FIG. 5 is a diagram illustrating attachment of the sensor assembly of the biomagnetic measurement apparatus according to the present embodiment;

FIGS. 6A to 6C are diagrams illustrating attachment of a magnetic sensor to the sensor assembly of the biomagnetic measurement apparatus according to the present embodiment;

FIG. 7 is a diagram illustrating attachment of the magnetic sensor to the sensor assembly of the biomagnetic measurement apparatus according to the present embodiment;

FIGS. 8A to 8C are diagrams for explaining a sensor holder of the biomagnetic measurement apparatus according to the present embodiment;

FIG. 9 is a diagram for explaining the sensor holder of the biomagnetic measurement apparatus according to the present embodiment;

FIG. 10 is a diagram for explaining measurement by the biomagnetic measurement apparatus according to the present embodiment;

FIG. 11 is a diagram for explaining measurement by the biomagnetic measurement apparatus according to the present embodiment; and

FIG. 12 is a diagram for explaining measurement by the biomagnetic measurement apparatus according to the present embodiment.

### DESCRIPTION OF THE EMBODIMENTS

Hereinafter, an embodiment for carrying out the present invention will be described with reference to the drawings. In the following drawings, the same components are indicated by the same reference numerals, and overlapping descriptions may be omitted.

Hereinafter, the biomagnetic measurement apparatus according to the present embodiment will be described in detail with reference to the accompanying drawings. The present invention is not limited by the present embodiment.

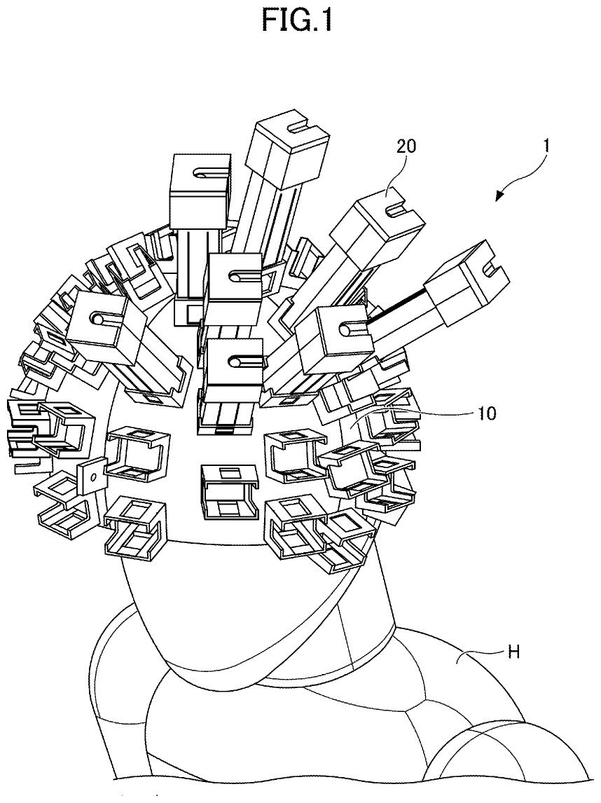

FIG. 1 is a diagram illustrating a state of use of a biomagnetic measurement apparatus 1, which is an example of the biomagnetic measurement apparatus according to the present embodiment. FIG. 1 is a diagram illustrating a state in which the biomagnetic measurement apparatus 1 is mounted to the head of a human body H, which is an object of measurement.

The biomagnetic measurement apparatus 1 according to the present embodiment is worn on the head of the human body H, which is the object of measurement. The biomagnetic measurement apparatus 1 includes a helmet-like mounting part 10 and a plurality of sensor assemblies 20.

In FIG. 1, as an example of measuring only the head region of interest, the sensor assemblies 20 are arranged only on a part of the head of the human body H. For example, the sensor assemblies 20 may be arranged on the entire head of the human body H. When the sensor assemblies 20 are arranged on the entire head of the human body H, the total number of the sensor assemblies 20 is about 100.

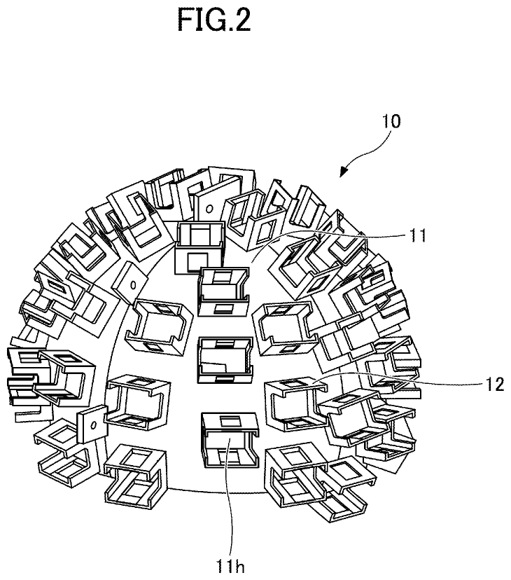

FIG. 2 is a diagram illustrating a mounting part 10 of the biomagnetic measurement apparatus 1, which is an example of the biomagnetic measurement apparatus according to the present embodiment. The mounting part 10 includes a base 11 and a plurality of sensor fixing parts 12. The mounting part 10 is formed by integrally forming the base 11 and a plurality of the sensor fixing parts 12. When the base 11 and the plurality of sensor fixing parts 12 are integrally formed, the positional accuracy of the sensor fixing part 12 can be increased.

The base 11 is a member for attaching a plurality of the sensor assemblies 20 to the head of the human body H. The subject wears the base 11 over the head. The base 11 has, for example, a hemispherical shape. The shape of the base 11 is not limited to the hemispherical shape. For example, the base 11 may have a half-ellipsoid shape.

The mounting part 10 includes a plurality of sensor fixing parts 12. The number of the sensor fixing parts 12 is about 100, for example. The base 11 has a plurality of openings 11h corresponding to the portion in which the sensor fixing parts 12 are located. Through each of the openings 11h, a magnetic sensor 30 provided within the sensor assembly 20 contacts the head of the human body H.

The mounting part 10 is formed by integrally forming the base 11 and a plurality of the sensor fixing parts 12. The mounting part 10 may be formed by manufacturing the base 11 and a plurality of the sensor fixing parts 12 using different members, and fixing the base 11 and the plurality of the sensor fixing parts 12 by fitting, screwing, bonding, or the like.

For example, the mounting part 10 is made entirely of a non-magnetic material. That is, the base 11 and a plurality of the sensor fixing parts 12 are made of a non-magnetic material. Specifically, the mounting part 10 is made of resin. When a non-magnetic material is used for the mounting part 10, magnetic noise can be eliminated. The magnitude of the magnetic field emitted from a living body such as a human body, which is an object of measurement, is on the order of femtotesla to picotesla, and is extremely weak. Therefore, it is desirable to eliminate external magnetic noise as much as possible.

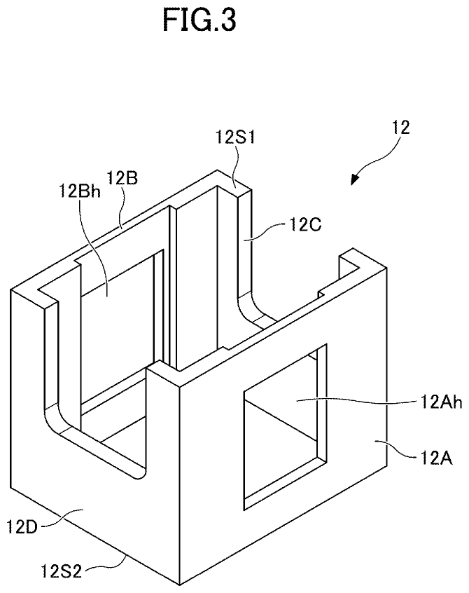

FIG. 3 is a diagram illustrating the sensor fixing part 12 of the biomagnetic measurement apparatus 1, which is an example of the biomagnetic measurement apparatus according to the present embodiment. The sensor fixing part 12 includes a wall 12A, a wall 12B, a wall 12C and a wall 12D. The wall 12A, the wall 12B, the wall 12C, and the wall 12D are connected in the order of the wall 12A, the wall 12C, the wall 12B, and the wall 12D.

The sensor fixing part 12 has a box-like shape having openings in the upper portion 1251 and lower portion 12S2 formed by the wall 12A, the wall 12B, the wall 12C, and the wall 12D. The wall 12A, the wall 12B, the wall 12C, and the wall 12D form the sides of the box. The upper portion 12S1 and the lower portion 12S2 of the sensor fixing part 12 are open.

The wall 12A has a rectangular opening 12Ah. The wall 12B has a rectangular opening 12Bh. Each of the openings 12Ah and 12Bh is engaged by protrusions 42a, which will be described later.

The area of the opening of the upper portion 12S1 may be larger than the area of the opening of the lower portion 12S2. The lower portion 12S2 connects to the base 11. The lower portion 12S2 is positioned corresponding to the opening 11h of the base 11. Accordingly, when a sensor holder 40 is fixed to the sensor fixing part 12, the magnetic sensor 30 can be pressed against the object of measurement.

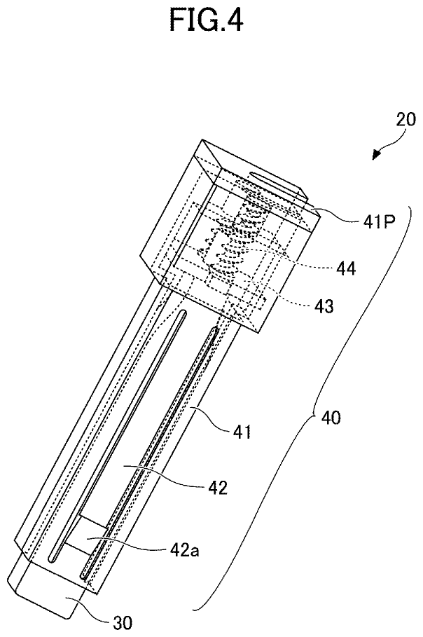

FIG. 4 is a diagram illustrating the sensor assembly 20 of the biomagnetic measurement apparatus 1, which is an example of the biomagnetic measurement apparatus according to the present embodiment. FIG. 4 is a diagram illustrating an exterior view of the sensor assembly 20. The sensor assembly 20 includes the magnetic sensor 30 and the sensor holder 40.

The magnetic sensor 30 is a sensor that detects a magnetic field from the human body H. The magnetic sensor 30 is, for example, an optically pumped atomic magnetometer (OPM). As the magnetic sensor 30, a magneto resistive (MR) sensor using the magneto resistance effect, and a magneto impedance (MI) sensor using the magneto impedance effect may be used. As the magnetic sensor 30, a nitrogen vacancy center (NVC) diamond sensor using a diamond nitrogen-vacancy center, or the like may be used.

The magnetic sensor 30 is used as a sensor in the present embodiment. The sensor is not limited to the magnetic sensor. Instead of the magnetic sensor, for example, a sensor for measuring a biological signal different from magnetic force, such as an electroencephalograph sensor may be used, as long as the sensor is attachable on the sensor fixing part 12.

The sensor holder 40 includes a cylindrical housing 41. The sensor holder 40 houses the magnetic sensor 30 inside the housing 41. The interior of the housing 41 of the sensor holder 40 has a shape similar to that of the magnetic sensor 30. One end of the housing 41 of the sensor holder 40 is a through hole penetrating from the inside. The magnetic sensor 30 is exposed to the outside through the through hole of the housing 41 of the sensor holder 40. The magnetic sensor 30 contacts the human body. H, which is the object of measurement.

The housing 41 of the sensor holder 40 includes a plate 42. The plate 42 includes protrusions 42a that engage with the opening 12Ah or the opening 12Bh of the sensor fixing part 12. Inside the housing 41, a coil spring 44 for pushing the magnetic sensor toward the human body H is provided. The sensor holder 40 includes a push plate 43 between the magnetic sensor 30 and the coil spring 44. The coil spring 44 is positioned between a plate 41P at the distal end of the housing 41 and the push plate 43. The coil spring 44 presses the magnetic sensor 30 against the object of measurement via the push plate 43.

The coil spring 44 is an example of a pressing part.

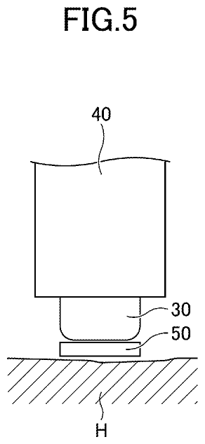

FIG. 5 is a diagram illustrating attachment of the sensor assembly 20 of the biomagnetic measurement apparatus 1, which is an example of the biomagnetic measurement apparatus according to the present embodiment. The magnetic sensor 30 held by the sensor holder 40 contacts the human body H, which is the object of measurement, via a buffer member 50.

The temperature of the magnetic sensor 30 may be above a body temperature. Accordingly, a person, which is the object of measurement, may feel the magnetic sensor 30 hot. Further, a person, which is the object of measurement, may feel uncomfortable depending on the hardness or shape of the material of the magnetic sensor 30. The magnetic sensor 30 of the biomagnetic measurement apparatus 1 according to the present embodiment contacts the human body H via the buffer member 50.

The buffer member 50 may be formed of, for example, an insulating material.

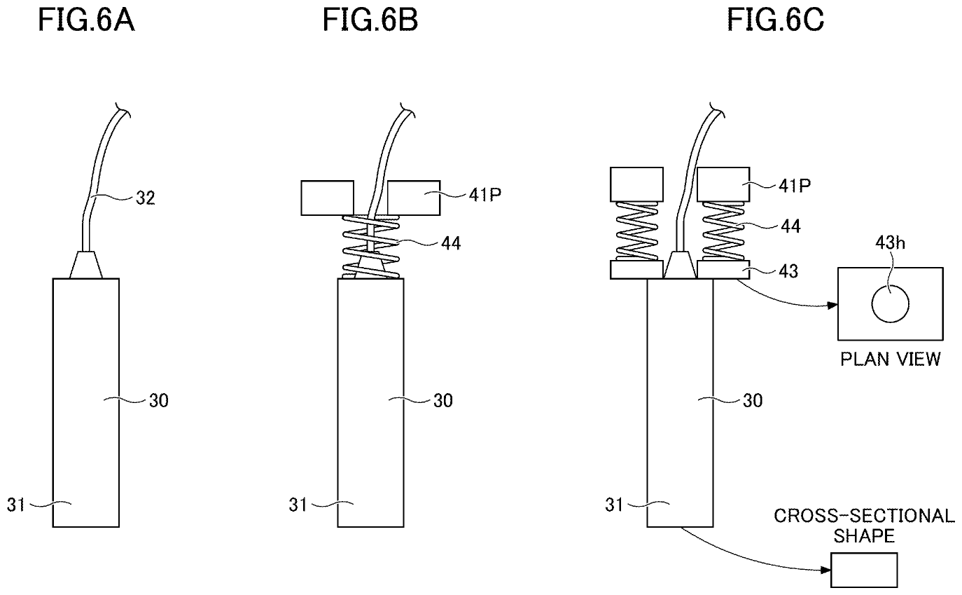

FIGS. 6A to 6C are diagrams illustrating attachment of the magnetic sensor 30 to the sensor assembly 20 of the biomagnetic measurement apparatus 1, which is an example of the biomagnetic measurement apparatus according to the present embodiment. The magnetic sensor 30 includes a sensor head 31 and a cable 32 (FIG. 6A).

The coil spring 44 pushes the magnetic sensor 30 from the cable 32 side to the object of measurement (FIG. 6B). When pushing the sensor head 31, the cable 32 may become a hindrance. In a case where the cable 32 becomes a hindrance, a push plate 43 is preferably used (FIG. 6C). The push plate 43 is preferably larger than the cross-sectional area of the magnetic sensor 30.

The push plate 43 includes an opening 43h in the center. The cable 32 penetrates the opening 43h. By pushing the push plate 43, the coil spring 44 pushes the magnetic sensor 30 toward the object of measurement.

Preferably, the coil springs 44 are provided on both sides of the opening 43h, for example. When the coil springs 44 are provided on both sides of the opening 43h, the magnetic sensor 30 can be pressed uniformly.

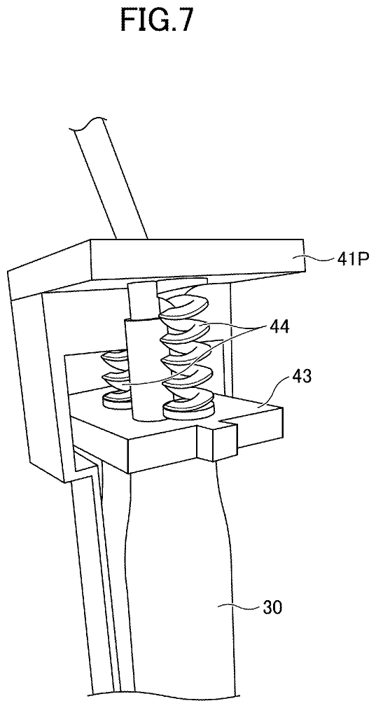

FIG. 7 is a diagram illustrating attachment of the magnetic sensor 30 to the sensor assembly 20 of the biomagnetic measurement apparatus 1, which is an example of the biomagnetic measurement apparatus according to the present embodiment. FIG. 7 illustrates the internal structure of the sensor holder 40. FIG. 7 illustrates the arrangement of the magnetic sensor 30, the push plate 43, and the coil spring 44. The sensor holder 40 includes two coil springs 44 on the front side and the rear side in FIG. 7.

The coil spring 44 and the push plate 43 keep pushing the magnetic sensor 30 toward the object of measurement. Therefore, misalignment of the magnetic sensor 30 with respect to the object of measurement does not occur. Further, when the coil spring 44 is used, the sensor holder 40 can stably exert a force and is excellent in durability as compared with a case in which the pressure is adjusted by air pressure or a case in which a rubber seal is used.

When the coil spring 44 is used, the spring constant of the coil spring 44 may be different depending on the object of measurement. By changing the spring constant of the coil spring 44, the force applied to the surface (a body surface) of the object of measurement can be changed.

The coil spring 44 is made of a non-magnetic material, such as a resin. When the coil spring 44 is formed with a non-magnetic material, magnetic noise can be substantially eliminated.

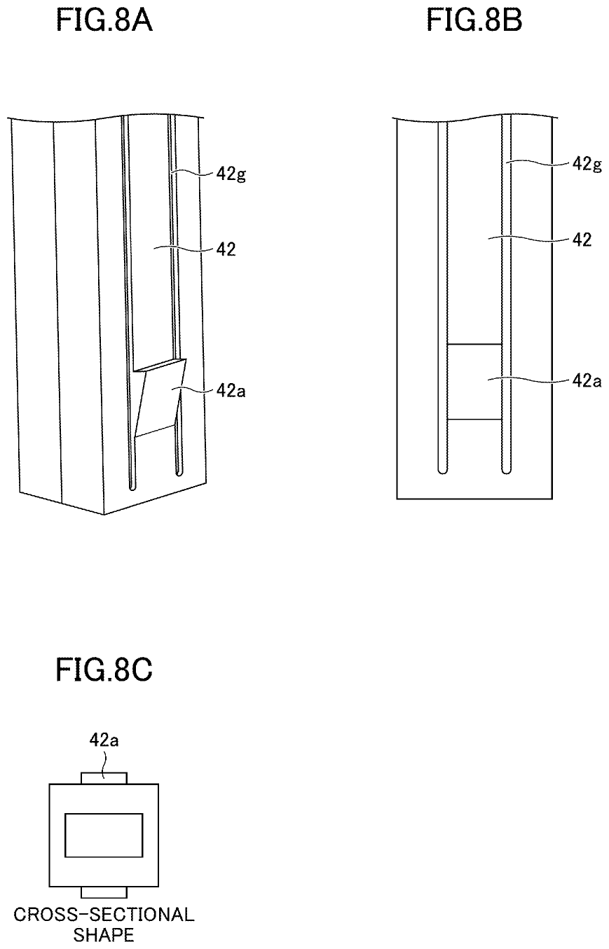

FIGS. 8A to 8C are diagrams for explaining the sensor holder 40 of the biomagnetic measurement apparatus 1, which is an example of the biomagnetic measurement apparatus according to the present embodiment. FIG. 8A is a perspective view of the sensor holder 40 of the biomagnetic measurement apparatus 1. FIG. 8B is a side view of the sensor holder 40 of the biomagnetic measurement apparatus 1. FIG. 8C is a bottom view illustrating the sensor holder 40 of the biomagnetic measurement apparatus 1 as viewed from the object of measurement side.

The sensor holder 40 of the biomagnetic measurement apparatus 1 includes the plate 42 in the housing 41. The plate 42 is made by providing the hollow housing 41 with two grooves 42g. The grooves 42g penetrate one side of the housing 41. The plate 42 is deformable by pressing with a human finger.

The plate 42 includes the protrusion 42a. The protrusion 42a has, for example, a wedge-like shape. The housing 41 including the plate 42 including the protrusion 42a can be easily and accurately manufactured using, for example, a three-dimensional printer. The housing 41 also includes the plate 42 and the protrusion 42a on the opposite side of the surface having the plate 42 illustrated in FIG. 8.

The protrusion 42a fits with the opening 12Ah or the opening 12Bh of the sensor fixing part 12. When the protrusion 42a fits with the opening 12Ah or the opening 12Bh of the sensor fixing part 12, the sensor holder 40 is fixed to the sensor fixing part 12.

When the protrusion 42a has a wedge-like shape, an operator attaching the sensor can insert the sensor holder 40 into the sensor fixing part 12. By inserting the sensor holder 40 into the sensor fixing part 12, the sensor holder 40 is mounted on the sensor fixing part 12. Also, by pushing and pulling the plate 42, the sensor holder 40 is detached from the sensor fixing part 12.

The sensor holder 40 is attached to the sensor fixing part 12 by fitting with the opening 12Ah or the opening 12Bh. Therefore, attachment and detachment of the sensor holder 40 and the sensor fixing part 12 can be performed in a several seconds of operation. Accordingly, even when the number of the magnetic sensors 30 increases, the magnetic sensors 30 can be attached and detached in a short period of time.

The cross-sectional shape inside the sensor holder 40 is rectangular. The cross-sectional shape inside the sensor holder 40 is rectangular shape that is to conform with the shape of the magnetic sensor 30. When the cross-sectional shape inside the sensor holder 40 is shaped to conform with the shape of the magnetic sensor 30, the magnetic sensor 30 can be stably accommodated.

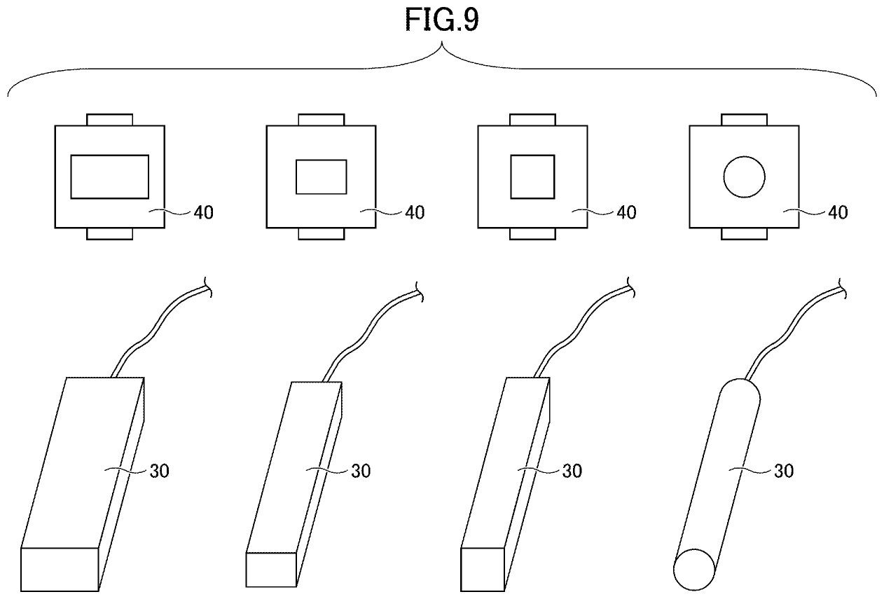

The outer shape and the size of the magnetic sensor 30 may differ depending on the measurement method. FIG. 9 is a diagram for explaining the sensor holder 40 of the biomagnetic measurement apparatus 1, which is the biomagnetic measurement apparatus according to the present embodiment. The upper views of FIG. 9 are views of the sensor holders 40 viewed from the object of measurement side. The lower views of FIG. 9 are perspective views illustrating the magnetic sensors 30 corresponding to each of the sensor holders 40.

The sensor holder 40 varies in shape and size of the inside of the sensor holder 40 in accordance with the shape of the magnetic sensor 30. For example, when the cross-section of the outer shape of the magnetic sensor 30 is circular, the cross-sectional shape inside the sensor holder 40 is circular.

The cross-sectional shape inside the sensor holder 40 is matched with the outer shape of the magnetic sensor 30. Accordingly, the base 11 and the sensor fixing part 12 may be manufactured as common parts, and the sensor holder 40 may be manufactured according to the type of the magnetic sensor 30.

In contrast, for example, it is not desirable to manufacture the base and the fixing part according to each of the plurality of types of magnetic sensors, from the viewpoint of resource savings and cost. In addition, it is not desirable to replace the base and the fixing part for each measurement, from the viewpoint of reducing man-hours and time.

In the biomagnetic measurement apparatus 1 according to the present embodiment, the cross-sectional shape inside the sensor holder 40 is changed in accordance with the magnetic sensor 30. Therefore, even when the magnetic sensor 30 is changed, the same mounting part 10, that is, the same base 11 and the same sensor fixing part 12 can be used.

Normally, in the biomagnetic measurement apparatus 1, a plurality of the same magnetic sensors are provided. In the sensor holder 40, different types of the magnetic sensors 30 can be used simultaneously by using multiple types of the sensor holders 40.

For example, in the measurement of brain magnetism, when it is desired to accurately measure only the region of interest, it is possible to use magnetic sensors with high sensitivity in the region, and magnetic sensors with normal sensitivity in other regions. When all of the magnetic sensors are magnetic sensors with high sensitivity, the cost will be high; in contrast, when some of the magnetic sensors are magnetic sensors with high sensitivity, the cost of the device will be reduced.

Some magnetic sensors have coils, inside the sensor, that cancel the magnetism. When such magnetic sensors are placed adjacent to each other, the magnetism emitted by the coil is measured by the adjacent magnetic sensors, and the accuracy of measurement is reduced. This effect is known as crosstalk. By alternately arranging magnetic sensors having a coil therein and magnetic sensors not having a coil, it is possible to measure biomagnetism without lowering the arrangement density of the sensors.

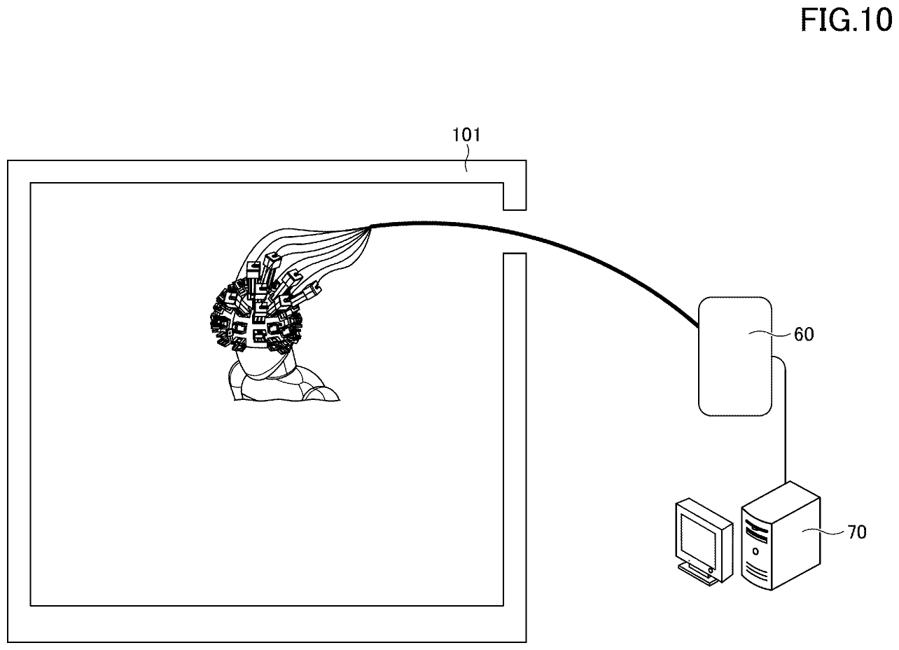

Next, the environment for measurement using the biomagnetic measurement apparatus 1, which is an example of the biomagnetic measurement apparatus according to the present embodiment, will be described. FIGS. 10 to 12 are diagrams for explaining the measurement by the biomagnetic measurement apparatus 1. The biomagnetic measurement apparatus 1 is an example of the biomagnetic measurement apparatus according to the present embodiment.

Biomagnetic measurement is usually performed in an environment where the environmental magnetic field, such as geomagnetic field, is reduced to a certain extent. For example, FIG. 10 is a diagram illustrating the measurement in a magnetically shielded room 101. A controller 60 performs control and data recording of the magnetic sensor 30. The magnetic sensor 30 connects to the controller 60 via a cable. The controller 60 is also connected, for example, to a computer 70. The computer 70 controls the entire biomagnetic measurement.

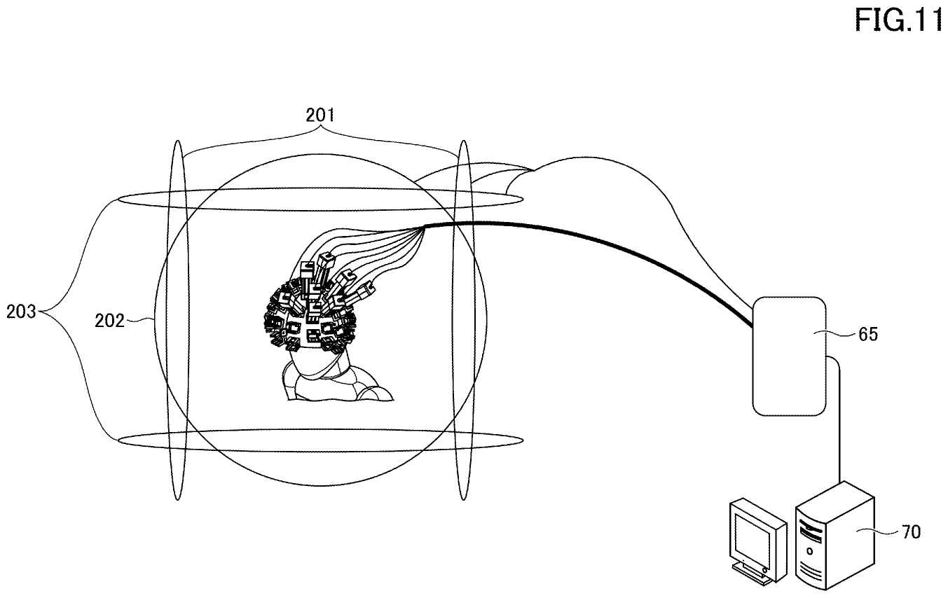

As illustrated in FIG. 11, a magnetic cancel coil 201, a magnetic cancel coil 202, and a magnetic cancel coil 203 may be used instead of the magnetically shielded room 101. For example, as the magnetic cancel coil, a Helmholtz coil is used. The magnetic cancel coil is controlled by a controller 65. The controller also performs control and data recording of the magnetic sensor 30. When magnetic cancel coils are used, an environment for measurement can be constructed at low cost.

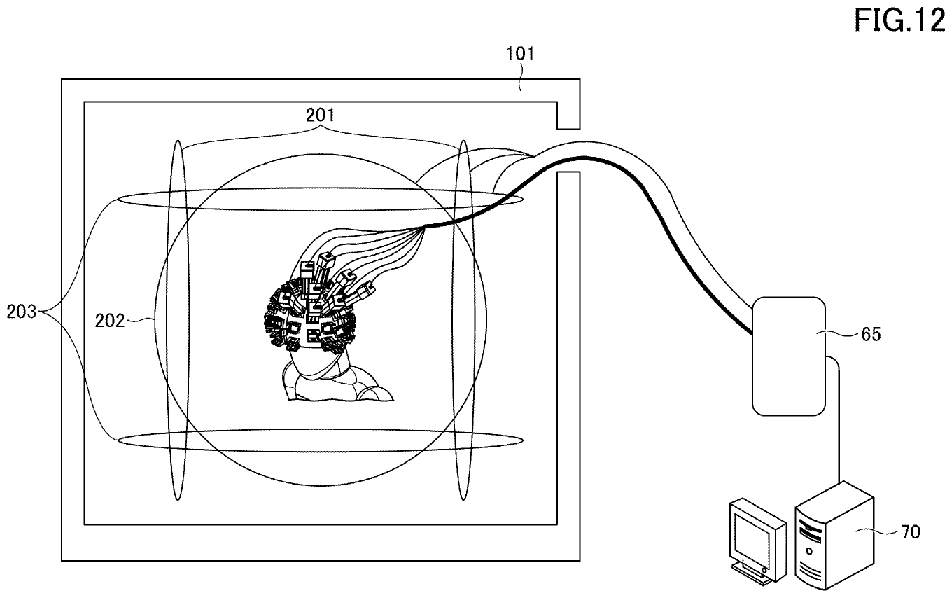

When more accurate measurement is desired, as illustrated in FIG. 12, the magnetic cancel coil 201, the magnetic cancel coil 202, and the magnetic cancel coil 203 may be placed in the magnetically shielded room 101.

In the biomagnetic measurement apparatus according to the present embodiment, because the magnetic sensor contacts the object of measurement, misalignment is prevented. In addition, the magnetic sensor can be easily and quickly attached and detached.

In the biomagnetic measurement apparatus 1, the sensor holder 40 houses the magnetic sensor 30. The sensor fixing part 12 integrally formed with the base 11 introduces inside and fixes the sensor holder 40 that houses the magnetic sensor 30. The sensor holder 40 includes the coil spring 44 for pushing the magnetic sensor toward the object of measurement. Because the coil spring 44 continuously pushes the magnetic sensor 30, misalignment is prevented. Also, because the sensor holder 40 and the sensor fixing part 12 are fitted, the sensor holder 40 can be fixed to the sensor fixing part 12.

The sensor holder 40 includes the plate 42 including the protrusion 42a. The sensor fixing part 12 includes the opening 12Ah and the opening 12Bh that are to be fitted with the protrusions 42a. By the plate 42 and the sensor fixing part 12, the sensor holder 40 is fixed to the sensor fixing part 12. Further, when the plate 42 is deformable by the force of pushing with a finger, the magnetic sensor 30 can be quickly and easily attached and detached.

In the biomagnetic measurement apparatus 1, when the base 11 and a plurality of the sensor fixing parts 12 are integrally formed, the positional accuracy of the sensor fixing parts 12 can be improved. Therefore, the positional accuracy of the magnetic sensor 30 when mounted can be improved.

The sensor holder 40 includes the push plate 43 for pushing the magnetic sensor 30 between the coil spring 44 and the magnetic sensor 30. The coil spring 44 presses the magnetic sensor 30 against the object of measurement via the push plate 43. The area of the push plate 43 is larger than that of the end face of the magnetic sensor 30 so that pressure is easily applied.

The sensor holder 40 includes two coil springs 44. The two coil springs 44 push from both sides of the magnetic sensor 30 via the push plate 43. Thus, a uniform force can be applied from the coil spring 44 to the magnetic sensor 30. Also, a uniform force can be applied to the object of measurement.

The base 11, the sensor fixing part 12, the sensor holder 40, and the coil spring 44 of the biomagnetic measurement apparatus 1 are made of a non-magnetic material. When the base 11, the sensor fixing part 12, the sensor holder 40, and the coil spring 44 of the biomagnetic measurement apparatus 1 are made of a non-magnetic material, magnetic noise can be reduced.

When the cross-sectional shape inside the sensor holder 40 is formed to match the outer shape of the magnetic sensor 30, a plurality of the magnetic sensors 30 having different shapes can be attached to the same fixing part.

In the present embodiment, a sensor mounted to the head of the human body H is used. However, the part measured by the sensor is not limited to the head. For example, sensors that measure biological signals of, for example, the abdomen, neck, shoulders, arms, and the like may be used.

The embodiments disclosed herein are exemplary in all respects and are not limiting. The embodiments described above may be omitted, substituted, or changed in various forms without departing from the appended claims and spirit thereof.

According to at least one embodiment, a biomagnetic measurement apparatus is provided that is capable of preventing misalignment of a magnetic sensor by bringing the magnetic sensor in contact with an object of measurement and enables easy attachment and detachment of the magnetic sensor.

What is claimed is:

1. A biomagnetic measurement apparatus comprising:
a magnetic sensor;
a sensor holder having a cylindrical shape and configured to accommodate and hold the magnetic sensor;
a sensor fixing part to which the sensor holder is fixed; and
a base to which the sensor fixing part is mounted and configured to be mounted to an object of measurement,
wherein the sensor holder includes: a pressing part configured to press the magnetic sensor toward the object of measurement; and a plate including a protrusion configured to engage with the sensor fixing part.

2. The biomagnetic measurement apparatus according to claim 1, wherein the sensor fixing part includes an opening that is configured to be fitted with the protrusion.

3. The biomagnetic measurement apparatus according to claim 1, wherein the base and the sensor fixing part are integrally formed.

4. The biomagnetic measurement apparatus according to claim 1, wherein the sensor holder includes a push plate between the magnetic sensor and the pressing part.

5. The biomagnetic measurement apparatus according to claim 4, wherein the pressing part includes two coil springs.

6. The biomagnetic measurement apparatus according to claim 1, wherein the base, the sensor fixing part, and the sensor holder are made of a non-magnetic material.

7. The biomagnetic measurement apparatus according to claim 1, wherein the sensor holder has a rectangular cross-sectional shape on an interior side thereof.

8. The biomagnetic measurement apparatus according to claim 1, wherein the sensor holder has a circular cross-sectional shape on an interior side thereof.
